(19) United States
(12) Patent Application Publication
(10) Pub. No.: US 2010/0009443 A1
(43) Pub. Date: Jan. 14, 2010

(54) Laboratory Apparatus for a Controlled Environment

(75) Inventor: Andrew Skinn, West Yorkshire (GB)

Correspondence Address: WADDEY & PATTERSON, P.C., 1600 DIVISION STREET, SUITE 500, NASHVILLE, TN, 37203, US

(73) Assignee: RUSKINN LIFE SCIENCES LIMITED, Bridgend (GB)

(21) Appl. No.: 12/264,465

(22) Filed: Nov. 4, 2008

(30) Foreign Application Priority Data
Jul. 8, 2008 (GB) 0812477.8

(51) Int. Cl. C12N 5/00; C12M 1/00

(52) U.S. Cl. 435/375; 435/303.1

(57) ABSTRACT

A controlled environment system for use in In-Vitro Fertilization (IVF) or other biological procedures and a method for conducting such a biological procedure. The system includes a main chamber, one or more manipulator devices for manipulating items in the interior of the chamber, a microscopy viewing system enabling viewing of the manipulation procedure, means for modifying the proportions of gas in a multi gas chamber atmosphere set up in the main chamber, chamber heater means for heating the gas atmosphere in the main chamber, a recirculation gas circuit providing for recirculation of the main chamber gas atmosphere, the recirculation gas circuit including a particulate filter arrangement and a sterilization arrangement.

### CROSS-REFERENCES TO RELATED APPLICATIONS

This application claims benefit of the following patent application which is hereby incorporated by reference: UK Patent Application No. 0812477.8 filed Jul. 8, 2008.

### BACKGROUND OF THE INVENTION

1. Field of the Invention

The present invention relates to a laboratory apparatus particularly suited for use in, but not limited for use in, In-Vitro Fertilization (IVF).

2. Description of the Prior Art

IVF treatment has gained popularity over the last twenty five years providing the opportunity for infertile couples to improve chances of reproduction. The female is treated with hormones such that a large number of unfertilized eggs can be extracted and taken to a laboratory where they are treated under carefully controlled conditions.

The unfertilized eggs are washed, sorted and fertilized and then transferred to an incubating environment which is arranged to as closely as possible resemble the conditions inside the body. For this reason, careful control is made of the temperature, humidity and gas concentrations. Once the fertilized eggs have reached embryonic stage, the best embryos are selected and implanted into the female.

As the conditions under which the embryo forms are critical and must closely resemble those found in the body, an apparatus must be provided that achieves and constantly maintains such a suitable environment in terms of temperature, humidity and concentration of mixed gasses. It is known that a high proportion of IVF failures are due to the effects of the environment, which is typically controlled in an active chamber. An example of such a chamber is disclosed in WO-A-2005/040330.

In so called Intracytoplasmic Sperm Injection (ICSI) techniques, a single sperm is injected directly into a respective egg. The procedure is typically carried out under a microscope using multiple micromanipulation devices (micromanipulators, microinjectors and micropipettes). A holding pipette stabilizes the selected oocyte and a needle is pierced through the oolemma and into the inner part of the oocyte. The distal end of the needle is then loaded with a single selected sperm and released into the oocyte.

Prior art controlled atmosphere IVF chambers an apparatus have not been suitable for use in certain IVF procedures, such as ICSI.

An improved apparatus for use in IVF procedures has now been devised.

### BRIEF SUMMARY OF THE INVENTION

According to a first aspect, the present invention provides a controlled environment system for use in IVF or other biological procedures, the system comprising:
a main chamber containing:
   one or more manipulator devices for manipulating items in the interior of the chamber, and
   a microscopy viewing system enabling viewing of the manipulation procedure;
means for modifying the proportions of gas in a multi gas chamber atmosphere set up in the main chamber;
chamber heater means for heating the gas atmosphere in the main chamber;
a recirculation gas circuit providing for recirculation of the main chamber gas atmosphere, the recirculation gas circuit including a particulate filter arrangement and a sterilization arrangement.

According to a second aspect, the present invention provides a method of conducting a biological procedure in a controlled environment in which an ICSI or another procedure for sperm injection is conducted on an oocyte in a controlled atmosphere of a chamber and subsequent incubation is carried out in the same chamber, the environment being controlled to have a predetermined gas mix atmosphere, a predetermined chamber temperature and the atmosphere being re-circulated via a recirculation loop extending outside the chamber which includes a sterilization function and a filter function.

It is preferred that the system includes recirculation gas circuit heater means for heating the gas in the recirculation gas circuit. This enables condensation within the recirculation circuit to be avoided, which is important to maintain a consistent atmosphere in the main chamber.

It is preferred that the method according to the invention is carried out using the system of the present invention.

It is envisaged that the recirculation gas heater could also act as the heater for the main chamber atmosphere, however in certain embodiments a separate main chamber heater will be utilized.

The chamber beneficially has a sealed entry port for the passage of items into and out of the main chamber. The system preferably includes an interlock chamber for the passage of items into and out of the main chamber.

Beneficially, the system includes an air agitation system for the main chamber to enable circulation of air within in the main chamber itself.

It is preferred that the chamber is provided with glove or gauntlet barrier ports, enabling the user to have hand access into the interior of the main chamber with a hand received in the barrier glove or gauntlet communicating via a wall of the main chamber.

In an embodiment particularly suited to the system of the present invention, the one or more manipulator devices may comprise an ICSI micro-injection device and/or other ICSI devices.

Beneficially the main chamber is provided with a humidification system for modifying the humidity of the main chamber atmosphere.

The recirculation gas circuit has a circuit path via the filter and the sterilization arrangement which is outside the main chamber.

The filter arrangement may comprise a medical application HEPA filter.

The sterilization arrangement may comprise a UV sterilization device.

The sterilization filter is provided flow-wise upstream of the filter arrangement in the gas recirculation circuit. This ensures that any microorganisms are killed before they enter the filter.

The invention will now be further described in a specific embodiment, by way of example only, and with reference to the accompanying drawings.

### BRIEF DESCRIPTION OF THE DRAWINGS

FIG. 1 is a schematic representation of a controlled environment system in accordance with the present invention; and

FIG. 2 is a side view of a part of the system of FIG. 1.

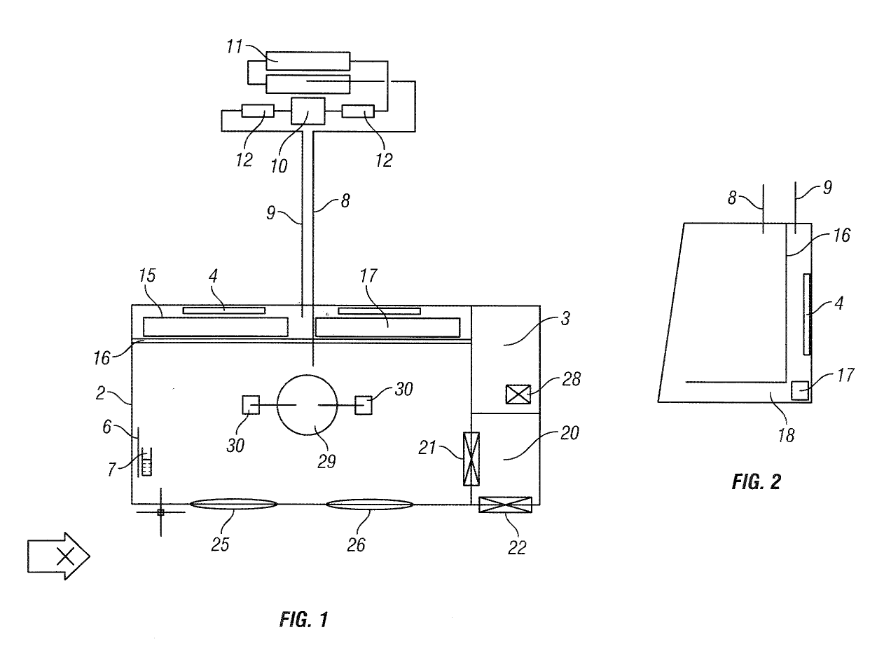

### DETAILED DESCRIPTION OF THE INVENTION

Referring to the drawings, there is shown a controlled environment apparatus 1 comprising a primary working chamber 2 for providing a controlled atmosphere for manipulation and/or culture of living biological material such as sperm, oocytes, or fertilized zygotes or embryos. The gas concentration proportion between oxygen, nitrogen and carbon dioxide is carefully monitored and controlled by a gas supply and mixer unit 3. For example the controller (not shown) such a programmable logic controller (PLC) may be set to maintain the chamber atmosphere gas mix at required predetermined concentration levels. A user interface device such as a user interactive control panel is typically provided to enable parameters to be manually reconfigured.

The chamber environment gas temperature is controlled by the PLC controller, in combination with a plate heater 4 mounted in a plenum 15 behind the rear wall 16 of the chamber. A duct 18, is provided at the bottom of the rear chamber wall 16 which in combination with a circulation fan 17 acts to draw gas from the main chamber 2, into the plenum 15 and past the heater to heat the air. Operation of the heater 4 and the circulation fan is controlled by the PLC controller.

Humidity in the chamber is also controlled by the PLC which controls operation of a humidifier 6 provided adjacent a water trough 7. Operation of the heater increases the humidity in chamber 2.

In this manner a controlled environment in terms of temperature, humidity and gas atmosphere mix can be maintained in the chamber 2. However for procedures in which fertilization and incubation are carried out in the same chamber, for example ICSI procedures envisaged to be carried out in the apparatus of the present invention, it is believed to be important to ensure that the air is filtered and sterilized, with appropriate care being taken to ensure that other characteristics of the atmosphere of the chamber 2 are maintained.

In accordance with the system of the present invention, the chamber atmosphere is drawn into a “closed loop” external recirculation circuit which leaves the chamber via sealed outlet 8 and is returned via sealed inlet 9. The recirculation loop is provided with a fan 10 to draw out the chamber atmosphere into the recirculation loop and direct it to an in-line ultraviolet light sterilization unit 11 and subsequently a HEPA filtration system 12 comprising a pair of HEPA filters arranged in series. This arrangement can be used to sterilize and to filter out particulates present (including bacteria and viruses). The light sterilization unit 11 is positioned flow-wise upstream of the HEPA filtration system 12 in order to ensure that bacteria/viruses etc. are killed of before entering the filtration system 12.

The UV and HEPA units can be replaced off-line without the need to enter the main chamber. The system ensures that the air re-circulated to the main chamber 2 is in accordance with the requirements of “Class B”.

The system is provided with an interlock chamber 20 having a sealed interlock port 21 in communication with the main chamber 2. A sealed interlock port 22 provides access into the chamber. The interlock chamber 20 may be arranged to evacuate environmental air and filled with the atmosphere of the chamber. An evacuation pump 28 is provided for this purpose. The interlocks 21, 22 can be set at a gas bleed setting to allow the interlock chamber 20 to fill with ambient air from outside the system, or the atmosphere of the chamber 2. In a preferred realization, when interlock door 22 is opened, ambient air will enter the interlock when the interlock door 22 is closed a pump is operated to vent atmosphere from the chamber 2 and exhaust the contents of interlock chamber 20 to atmosphere. The interlocks 21, 20 operated in sequence will enable the impact of external ambient conditions to have minimal impact on the atmosphere of the chamber when objects are brought into the main chamber 2 via the interlock 20.

An inspection microscope 29 is positioned internally of the main chamber 2, connected to a visual display screen which may be positioned outside the main chamber or more beneficially from a user perspective, internally of the chamber. A manipulator device such as an ICSI micro-injector device 30 is positioned in the interior of the main chamber 2. The microscopy system enables the user to operate the micro manipulator device 30 in an accurate and controlled manner.

The front wall of the main chamber is typically transparent and provided with a pair of gloved ports 25 26, into which a person can insert their hands and arms in order to facilitate manipulation of the micro devices (such as micro-injection device 30) and other items within the interior of the main chamber 2 whilst still providing a sealed barrier between the interior of the chamber and the chamber exterior environment.

In use for the ICSI process, the introduction of the sperm into the oocyte can take place in the controlled environment of the main chamber 1 in which the atmosphere is continuously controlled in terms of temperature, humidity and chamber gas atmosphere mix. Also the atmosphere is re-circulated via a filter and sterilization route to meet predetermined standards (for example “Class B” air standard). The microscope and micromanipulators present in the chamber 2 are used for carrying out the procedure. Following cytoplasm injection the zygotes can be maintained in the environment of the main chamber and development monitored. There is thus no need to have sperm injection and subsequent incubation carried out in separate environments or atmospheres. This enhances the chances of viable embryos forming.

Thus, although there have been described particular embodiments of the present invention of a new and useful Laboratory Apparatus for a Controlled Environment it is not intended that such references be construed as limitations upon the scope of this invention except as set forth in the following claims.

What is claimed is:

1. A controlled environment system for use in IVF or other biological procedures, the system comprising:
a main chamber containing:
one or more manipulator devices for carrying out a manipulating procedure internally of the chamber, and
a microscopy viewing system enabling viewing of the manipulation procedure;
a gas supply system including a gas modification arrangement for modifying the relative proportions of gases in a multi gas chamber atmosphere set up in the main chamber; and a recirculation gas circuit providing for recirculation of the main chamber gas atmosphere, the recirculation gas circuit including a particulate filter arrangement and a sterilization arrangement;
a chamber heater arrangement for heating the gas atmosphere in the main chamber.

2. A system according to claim 1, wherein the system includes a recirculation gas circuit heater system for heating the gas in the recirculation gas circuit.

3. A system according to claim 1, wherein the chamber has a sealed entry port for the passage of items into and out of the main chamber.

4. A system according to claim 1, wherein the system includes an interlock chamber for the passage of items into and out of the main chamber.

5. A system according to claim 1, wherein the system includes an air agitation system for the chamber to enable circulation of air within in the main chamber.

6. A system according to claim 1, wherein the chamber is provided with glove or gauntlet barrier ports, enabling the user to have hand access into the interior of the main chamber with a hand received in the barrier glove or gauntlet communicating via a wall of the main chamber.

7. A system according to claim 1, wherein the one or more manipulator devices comprise an ICSI micro-injection device.

8. A system according to claim 1, wherein the main chamber is provided with a humidification system for modifying the humidity of the main chamber atmosphere.

9. A system according to claim 1, wherein the recirculation gas circuit has a circuit path via the filter and the sterilization arrangement which is outside the main chamber.

10. A system according to claim 1, wherein the filter arrangement comprises a HEPA filter.

11. A system according to claim 1, wherein the sterilization arrangement comprises a UV sterilization device.

12. A system according to claim 1, wherein the sterilization filter is provided flow-wise upstream of the filter arrangement in the gas recirculation circuit.

13. A system according to claim 1, wherein the manipulator device and the microscopy viewing system are actuatable from externally of the chamber, whilst seated in the chamber.

14. A method of conducting a biological procedure in a controlled environment in which an ICSI or another procedure for sperm injection is conducted on an oocyte in a controlled atmosphere of a chamber and subsequent incubation is carried out in the same chamber, the environment being controlled to have a predetermined gas mix atmosphere, a predetermined chamber temperature and the atmosphere being re-circulated via a recirculation loop extending outside the chamber which includes a sterilization function and a filter function.
